(19) United States
(12) Patent Application Publication
(10) Pub. No.: US 2003/0108536 A1
(43) Pub. Date: Jun. 12, 2003

(54) Inhibition of graft versus host disease

(76) Inventor: David Spaner, Ontario (CA)

Correspondence Address: BURNS DOANE SWECKER & MATHIS L L P, POST OFFICE BOX 1404, ALEXANDRIA, VA, 22313-1404, US

(21) Appl. No.: 10/181,441

(22) PCT Filed: Jan. 26, 2001
(86) PCT No.: PCT/CA01/00076

(30) Foreign Application Priority Data
Jan. 28, 2000 (CA) 2,297,448

(51) Int. Cl. A61K 38/44; A61K 31/355; A61K 31/095; A61K 45/00

(52) U.S. Cl. 424/93.71; 424/94.4; 604/20; 514/458; 514/706

(57) ABSTRACT

The development of graft versus host disease in a mammalian patient undergoing cell transplantation therapy for treatment of a bone marrow mediated disease, is prevented or alleviated by subjecting at lest the T-cells of the allogeneic cell transplantation composition, in admixture with red blood cells of the donor, extracorporeally, to oxidative stress, in appropriate dosage amounts, such as bubbling a gaseous mixture of ozone and oxygen through a suspension of the T-cells. The process may also include irradiation of the cells with UV light, simultaneously with the application of the oxidative stress. The oxidative stress induces reduced inflammatory cytokine production and a reduced proliferative response in the T-cells.

### FIELD OF THE INVENTION

This invention relates to cellular compositions useful in medical treatments, processes for their preparation and their uses in medical treatments. More specifically, it relates to cellular compositions useful in alleviation of complications following allogeneic bone marrow transplantation, namely graft versus host disease in mammalian patients, especially in human patients, and to processes for preparation of such compositions of matter.

### BACKGROUND OF THE INVENTION

Bone marrow transplantation, BMT, is indicated following a process which destroys bone marrow. For example, following intensive systemic radiation or chemotherapy, bone marrow is the first target to fail. Metastatic cancers are commonly treated with very intensive chemotherapy, which is intended to destroy the cancer, but also effectively destroys the bone marrow. This induces a need for BMT. Leukemia is a bone marrow malignancy, which is often treated with BMT after chemotherapy and/or radiation has been utilized to eradicate malignant cells. BMT is currently used for treatment of leukemias which are life-threatening. Some autoimmune diseases may be severe enough to require obliteration of their native immune systems which includes concomitant bone marrow obliteration and requires subsequent bone marrow transplantation. Alleviation of any but the most acute life-threatening conditions involving bone marrow disorders with BMT is, however, generally regarded as too risky, because of the likelihood of the onset of graft versus host disease.

Graft-versus-host disease, GVHD, is an immunological disorder that is the major factor that limits the success and availability of allogeneic bone marrow or stem cell transplantation (collective referred to herein as allo-BMT) for treating some forms of otherwise incurable hematological malignancies, such as leukemia. GVHD is a systemic inflammatory reaction which causes chronic illness and may lead to death of the host mammal. At present, allogeneic transplants invariably run a severe risk of associated GVHD, even where the donor has a high degree of histocompatibility with the host.

GVHD is caused by donor T-cells reacting against systemically distributed incompatible host antigens, causing powerful inflammation. In GVHD, mature donor T-cells that recognize differences between donor and host become systemically activated. Current methods to prevent and treat GVHD involve administration of drugs such as cyclosporin-A and corticosteroids. These have serious side effects, must be given for prolonged periods of time, and are expensive to administer and to monitor. Attempts have also been made to use T-cell depletion to prevent GVHD, but this requires sophisticated and expensive facilities and expertise. Too great a degree of T-cell depletion leads to serious problems of failure of engraftment of bone marrow stem cells, failure of hematopoietic reconstitution, infections, or relapse. More limited T-cell depletion leaves behind cells that are still competent to initiate GVHD. As a result, current methods of treating GVHD are only successful in limited donor and host combinations, so that many patients cannot be offered potentially life-saving treatment.

### BRIEF REFERENCE TO THE PRIOR ART

U.S. Pat. No. 5,980,954 Bolton, issued Nov. 9, 1999, describes an autovaccine for alleviating the symptoms of an autoimmune disease in a mammalian patient, comprising an aliquot of modified blood obtained from the same patient and treated extracorporeally with ultraviolet radiation and an oxygen/ozone gas mixture bubbled therethrough, at an elevated temperature (42.5° C.), the autovaccine being re-administered to the same patient after having been so treated. This is a vaccination-type procedure, using modified autologous cells to treat autoimmune disorders in the patient.

It is an object of the present invention to provide a novel process of alleviating the development of GVHD complications in a mammalian patient undergoing allo-BMT procedures, i.e. using allogeneic cells.

It is a further object of the invention to provide a novel composition of allogeneic T-cells suitable for administration to a patient, in association with allogeneic stem cells in BMT procedures.

### SUMMARY OF THE INVENTION

According to the present invention, a patient being treated by allo-BMT is administered a composition containing T-cells obtained from an allogeneic donor, said T-cells having been subjected in vitro to modification and/or stress in the presence of a protective amount of biocompatible antioxidant, for example a biocompatible protectant selected from red blood cells, vitamin E, catalase and 2-mercaptoethanol, to induce therein decreased inflammatory cytokine production coupled with reduced proliferative responses. It appears that such modified and/or stressed allogeneic T-cells, when injected into a mammalian patient, have a down-regulated immune response and a down-regulated destructive allogeneic response against the recipient, so that engraftment of the hematopoietic stem cells, administered along with or separately from the stressed T-cells, can take effect with significantly reduced risk of development of GVHD. The population of stressed T-cells nevertheless appears to be able to exert a sufficient protective effect on the mammalian system to guard against failure of engraftment and against infection, whilst the hematopoietic system is undergoing reconstitution, at least in part, by proliferation and differentiation of the allogeneic stem cells. The biocompatible antioxidant serves to protect the T-cells from deleterious effects of oxidative stress during the in vitro modification, and ensures the production of appropriately modified, viable cells in sufficient numbers for effective use in BMT with reduced risk of development of GVHD.

One aspect of the present invention provides, accordingly, a process of treating a mammalian patient for alleviation of a bone marrow mediated disease, with alleviation of consequently developed graft versus host disease (GVHD), which comprises administering to the patient allogeneic hematopoietic stem cells and allogeneic T-cells, at least a portion of said T-cells having been modified and/or stressed in vitro in the presence of an effective amount of a biocompatible antioxidant, so as to induce an altered cytokine production profile and a reduced proliferative response therein, prior to administration to the patient.

Another aspect of the present invention provides a population of mammalian T-cells, essentially free of stem cells, said T-cells having been modified and/or stressed in vitro in the presence of a biocompatible antioxidant so as to induce in said cells an altered cytokine production profile and a reduced proliferative response.

A further aspect of the present invention provides a process for preparing an allogeneic cell population for administration to a human patient suffering from a bone marrow mediated disease, which comprises subjecting, in vitro, a population of donor cells enriched in T-cells to stress in the presence of an effective amount of a biocompatible antioxidant, to induce in said T-cells an altered cytokine production profile and a reduced proliferative response.

### DESCRIPTION OF THE PREFERRED EMBODIMENTS

The preferred biocompatible antioxidant for use in the present invention is a biocompatible protectant such as vitamin E, catalase or 2-mercaptoethanol, or more preferably, red blood cells, obtained from the same donor as the T-cells to be treated. The red blood cells, which contain compounds which exhibit antioxidative properties, are suitably used in amounts up uo 50% of the number of T-cells being subjected to oxidative stress, preferably in amounts from about 3-25%, by number. The presence of the donor red blood cells during the subjection to oxidative stress helps to protect the T-cells from deleterious effects of the oxidative stress such as excessive cell death, and generally modulates the severity of the oxidative stress applied to the T-cells, in a beneficial manner. Thus the presence of red blood cells of the donor in the suspension at the time of subjection of the suspension to stress serves to protect the T-cells of the PBMCs from total destruction or deactivation, and to ensure that enough T-cells survive, even in quiescent or senescent condition, to allow for subsequent activation and proliferation of surviving T-cells for reconstitution of an effective immune system in the patient/recipient.

The process of the present invention involves an initial collection of hematopoietic stem cells and T-cells from a donor. The preferred source of such cells is mobilized stem cells and T-cells from the peripheral blood of the donor, i.e., peripheral blood mononuclear cells (PBMCs). Stem cells are present in very small quantities in peripheral blood, and one preferred way of operating in accordance with the invention is to enrich the stem cell population of the donor's peripheral blood, and then to extract the donor's peripheral blood for use as a source of stem cells and T-cells for mixing with an appropriate quantity of biocompatible antioxidant and treatment of the resultant cell composition as described, and subsequent injection into the patient. Enrichment may be achieved by giving the donor a course of injections of appropriate growth factors, over several days e.g. five days prior to extracting peripheral blood from the donor. Appropriate cell fractions can be collected from the blood by leukapheresis, a known procedure, as it is extracted, with the plasma and red cells being returned to the donor, in a closed flow system. The white cell collection, which contains the stem cells (up to about 3%) and T-cells (about 40%) along with B-cells (20-30%), monocytes (20-30%) and other white cells, may be treated to alter the cytokine production profiles of the T-cells and to reduce the proliferative response of the T-cells, in admixture with biocompatible antioxidant, and then administered to the host patient, in accordance with the invention, as a whole collection of cells (peripheral blood m mononuclear cells). Alternatively, the donor T-cells are separated from the other cells, so that only the T-cells in admixture with the biocompatible antioxidant are subjected to stress and then administered to the patient, with the stem cells for engraftment being administered to the patient separately from the treated T-cells. For practical purposes, however, subjection of the collection of peripheral blood mononuclear cells in admixture with red blood cells of the donor, to the stressors is satisfactory, without further fractionation to isolate the T-cells. Separate administration of stem cells is preferred.

In an alternative, but less preferred, procedure, whole bone marrow of the donor can be used as the source of T-cells and stem cells for the process for the invention. Whole bone marrow has in the past been the usual source of cells for allogeneic cell transplantation procedures, and can indeed be used in the present process. It is however an inconvenient and uncomfortable procedure for the donor, requiring anaesthetic and lengthy extraction procedures. Any source of T-cells and stem cells from the donor can be used in principle, but peripheral blood enriched in stem cells and T-cells, in admixture with biocompatible antioxidant, is the most clinically convenient.

The alteration in cytokine production profile induced in the T-cells in the process of the invention is preferably a reduction in production of inflammatory cytokines, such as interferon-γ and tissue necrosis factor-α, and/or an increase in the production of anti-inflammatory cytokine(s) such as IL-10. This alteration is normally induced by subjection of the cells to oxidative stress.

The stress required to induce alteration in the cytokine profile of the T-cells is preferably oxidative stress, and may be applied to the T-cells and red blood cells by subjecting them, in the presence of biocompatible antioxidant, to an oxidative environment such as the addition of a gaseous, liquid or solid chemical oxidizing agent (ozone, molecular oxygen, ozone/oxygen gas mixtures, permanganates, periodates, peroxides, drugs acting on biological systems through an oxidative mechanism such as adriamycin, and the like). In one preferred method according to the invention, the T-cells are subjected, in suspension and in admixture with red blood cells, to a gaseous oxidizing agent, such as an ozone/oxygen gas mixture bubbled through the suspension of cells, optionally in combination with the simultaneous subjection of the cells to ultraviolet radiation, in appropriate doses.

One preferred method of subjecting the allogeneic T-cells to oxidative stress according to the invention involves exposing a suspension of the cells in admixture with red blood cells of the donor to a mixture of medical grade oxygen and ozone gas, for example by bubbling through the suspension a stream of medical grade oxygen gas having ozone as a minor component therein. The suspending medium may be any of the commonly used biologically acceptable media which maintains cells in viable condition. The ozone gas may be provided by any conventional source known in the art. Suitably the gas stream has an ozone content of from about 1.0-100 μg/ml, preferably 3-70 μg/ml and most preferably from about 5-50 μg/ml. The gas stream is supplied to the aliquot at a rate of from about 0.01-2 litres per minute, preferably 0.05-1.0 litres per minute, and most preferably at about 0.06-0.30 litres per minute (STP).

Another method of subjecting the T-cells to oxidative stress to render them suitable for use in the present invention is to add to a suspension of the cells and the biocompatible antioxidant a chemical oxidant of appropriate biological acceptability, and in biologically acceptable amounts. Permanganates, periodates and peroxides are suitable, when used in appropriate quantities. Hydrogen peroxide is particularly useful in demonstrating the effectiveness of the process of the invention in vitro and in giving guidance on the appropriate quantity of oxidizing agent to be used, although it may not be an agent of first choice for the present invention, in clinical applications. Thus, a suitable amount of oxidizing agent is hydrogen peroxide in a concentration of from 1 micromolar-2 millimolar, contacting a 10 ml suspension containing from 10⁶ to 10⁸ cells per ml, for 20 minutes, or equivalent oxidative stress derived from a different oxidizing agent. Optimum is about 1 millimolar hydrogen peroxide in such a suspension for about 20 minutes, or the equivalent of another oxidizing agent calculated to give a corresponding degree of oxidative stress to-the cells.

The size of the cell suspension to be subjected to oxidative stress is generally from about 0.1 ml to about 1000 ml, preferably from about 1-500, and containing appropriate numbers of T-cells for subsequent administration to a patient undergoing allo-BMT. These numbers generally correspond to those used in prior methods of allogeneic T-cell administration in connection with allo-BMT, and are familiar to those skilled in the art. Typically, the suspension is plasma containing from about 2 million to 5 million T-cells per 10 mls, e.g. 10⁸ PBMNCs, and from 2-10% red blood cells.

One specific process according to the invention is to subject the cell suspension simultaneously to oxygen/ozone bubbled through the suspension and ultraviolet radiation. This also effects the appropriate changes in the nature of the T-cells. Care must be taken not to utilize an excessive dosage of oxygen/ozone or UV, to the extent that the cell membranes are caused to be disrupted, or other irreversible damage is caused to an excessive number of the cells. The donor red blood cells in the cell suspension serve to ensure this, provided ordinary skill in the art is employed in conducting the process, and allow a simple, straightforward and rapid administration of the oxidative stress.

The temperature at which the T-cell suspension is subjected to the oxidative stress should be appropriate to keep the suspension in the liquid phase and should not be so high that it causes cell membrane disruption. The temperature should not be higher than about 45° C.

When ultraviolet radiation is used in conjunction with the oxygen/ozone oxidative stressor, it is suitably applied by irradiating the suspension under treatment from an appropriate source of UV radiation, while the aliquot is maintained at the aforementioned temperature and while the oxygen/ozone gaseous mixture is being bubbled through the aliquot. The ultraviolet radiation may be provided by any conventional source known in the art, for example by a plurality of low-pressure Ultraviolet lamps. Preferred UV sources are UV lamps emitting UV-C band wavelengths, i.e. at wavelengths shorter than about 280 nm. Ultraviolet light corresponding to standard UV-A (wavelengths from about 315 to about 400 nm) and UV-B (wavelengths from about 280 to about 315) sources can also be used. For example, an appropriate dosage of such UV light, applied simultaneously with the aforementioned temperature and oxidative environment stressors, can be obtained from lamps with a power consumption of from about 15 to about 30 watts and useful UV output of about 5-10 watts, arranged to surround the sample container holding the aliquot. Up to eight such lamps surrounding the sample bottle, operated at an intensity to deliver a total UV light energy at 253.7 nm at the surface of the blood of from about 0.025 to about 10 joules/cm², preferably from about 0.1 to about 3.0 joules/cm², may advantageously be used. Such a treatment provides a suspension aliquot which is appropriately modified according to the invention ready for injection into the patient. The red blood cells in the suspension serve to protect against cell damage resulting from possible administration of excess UV radiation.

The time for which the cell suspension is subjected to the stressors can be from a few seconds to about 60 minutes. It is normally within the time range of from about 0.5-60 minutes. This depends to some extent upon the chosen intensity of the UV irradiation, the temperature and the concentration of and rate at which the oxidizing agent is supplied to the aliquot. Some experimentation to establish optimum times and dosages may be necessary on the part of the operator, once the other stressor levels have been set. Under most stressor conditions, preferred times will be in the approximate range of about 0.5-10 minutes, most preferably 2-5 minutes, and normally around 3 minutes.

In the practice of one preferred process of the present invention, the suspension of cells may be treated with oxygen/ozone gas mixture and optionally also with UV radiation using an apparatus of the type described in U.S. Pat. No. 4,968,483 Mueller. The suspension is placed in a suitable, sterile, UV-radiation-transmissive container, which is then fitted into the machine. The temperature of the aliquot is adjusted to the predetermined value, e.g. 42.5±1° C., by the use of a suitable heat source such as an IR lamp, and the UV lamps are switched on for a fixed period before the gas flow is applied to the aliquot providing the oxidative stress, to allow the output of the UV lamps to stabilize. The oxygen/ozone gas mixture, of known composition and control flow rate, is applied to the aliquot, for the predetermined duration of 0.5-60 minutes, preferably 1-5 minutes and most preferably about 3 minutes as discussed above. In this way, the suspension is appropriately modified according to the present invention sufficient to achieve the desired effects of alleviation or prevention of GVHD.

From another aspect, the preferred embodiment of the present invention may be viewed as a process of treating allogeneic T-cells prior to their introduction into a patient, by extracorporeally stressing the T-cells, which comprises subjecting the T-cells in admixture with red blood cells of the donor to oxidative stress such as exposure to ozone or ozone/oxygen. The treated allogeneic T-cells from the process of the invention have a direct effect on the development and progression of GVHD. The donor T-cells pretreated according to the process of the invention prior to introduction into the host patient, have been modified, so that they no longer mount a deleterious response. Their ability to mount an inflammatory cytokine response has been decreased. For example their ability to secrete IFNγ, TNFα and IL-2, and their proliferative response to standard mitogens has been reduced. In some cases also, their ability to secrete anti-inflammatory cytokines such as IL-10 is increased. Accordingly they no longer react against incompatible systemically distributed host histocompatibility antigens to cause inflammation to any great extent. The allogeneic stem cells administered to the patient can proceed with engraftment with improved chance of success. After a period of time, the treated T-cells largely recover their proliferative ability and immune response functions, but remain relatively unresponsive (tolerant) to differing host histocompatibility antigens.

One process of the present invention may involve a step of ageing the stressed T-cells, at reduced temperature, prior to their administration to the recipient/patient, with the result that more T-cells appear to be rendered quiescent or senescent, and the period of time for such cells to restore normal function after injection into the patient is extended. Such an ageing step is not, howevere, necessary, and can complicate the procedure to an undesirable extent, in a clinical environment.

A preferred process according to the invention thus involves the successive steps of collection of a peripheral blood sample from the donor, and separation thereof to obtain the T-cell-containing PBMC fraction, dilution of the fraction with plasma of the donor to the appropriate concentration and addition of the appropriate amount of red cells. Then the suspension so formed is subjected to the oxidative stressor, in the form of an oxygen/ozone mixture bubbled through the suspension at a temperature of 42.5° C., optionally while under irradiation with UV light, as described above. The suspension is then washed, further diluted, and is then ready for administration to a patient/recipient, in conjunction with, but separately from, allogeneic stem cells.

The invention is further described, for illustrative purposes, in the following specific examples.

The spleen of a mammal offers a convenient, accessible source of cells, especially T-cells but also including small quantities of stem cells and is particularly useful in connection with animal models for experimental purposes.

Experimental testing to obtain indication of the utility of the process of the present invention was conducted using a model of acute GVHD in SCID mice. T-cells from C57B1/6J (B6) mice were intravenously injected into sub-lethally irradiated CB-17 SCID mice. The latter are congenitally lymphopenic and provide a strong stimulus for donor cells due to their complete disparity at the major histocompatibility locus (MHC). The mean survival time of host mice in this model is 14 days. GVHD is characterized by suppression of host hematopoietic recovery from irradiation; expansion of T-cells that use Vβ3 chain to form their T-cell receptor complexes (TCR's); spontaneous secretion of interferon-γ and TNF-α, by donor T-cells, and aberrant localization of donor T-cells to the red pulp areas of the spleen. If donor marrow is co-injected with T-cells, a chronic form of GVHD results.

### EXAMPLE 1 (COMPARATIVE)

Murine B6 spleen cells suspended in 100% fetal calf serum (FCS) were subjected to UV-oxidation-heat treatment. The cell suspension was subjected simultaneously to ultraviolet radiation from UV-C lamps, wavelength 253.7 nm, whilst bubbling through the suspension a gas mixture of 14-15 mcg/ml ozone/medical grade oxygen, at 42.5° C. The treatment took place for 3 minutes. Varying numbers were injected into sub-lethally irradiated CB-17 SCID mice. Their subsequent behaviour was compared with similar numbers of B6 spleen cells, not subjected to the treatment.

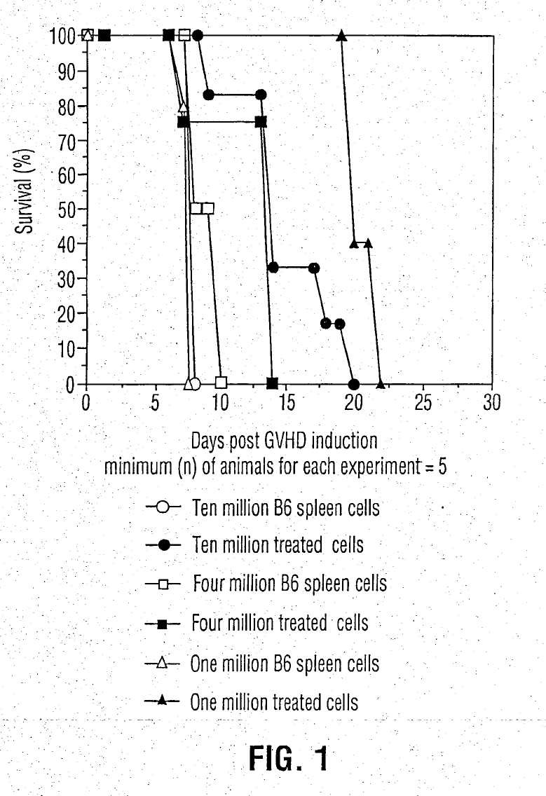

FIG. 1 is a graphical presentation of the results of these experiments, where the % survival of the animals in each group is plotted as ordinate against days following injection of the treated or untreated cells. At all dosage levels, there is a marked improvement of survival when the treated cells are used as opposed to the untreated cells, demonstrating potential for the process of the invention in alleviating GVHD.

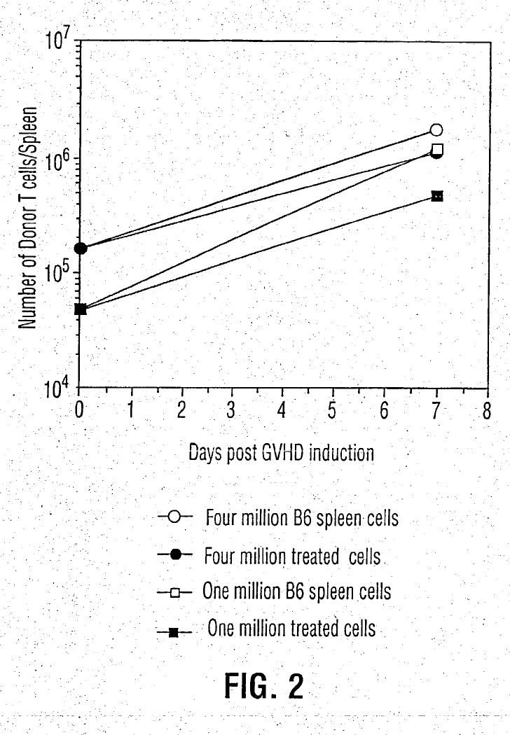

FIG. 2 of the accompanying drawings is a plot of the number of donor T-cells per spleen against days after GVHD induction, in these same experiments. This shows that the treated donor T-cells survive and expand in number in the host mice.

### EXAMPLE 2 (COMPARATIVE)

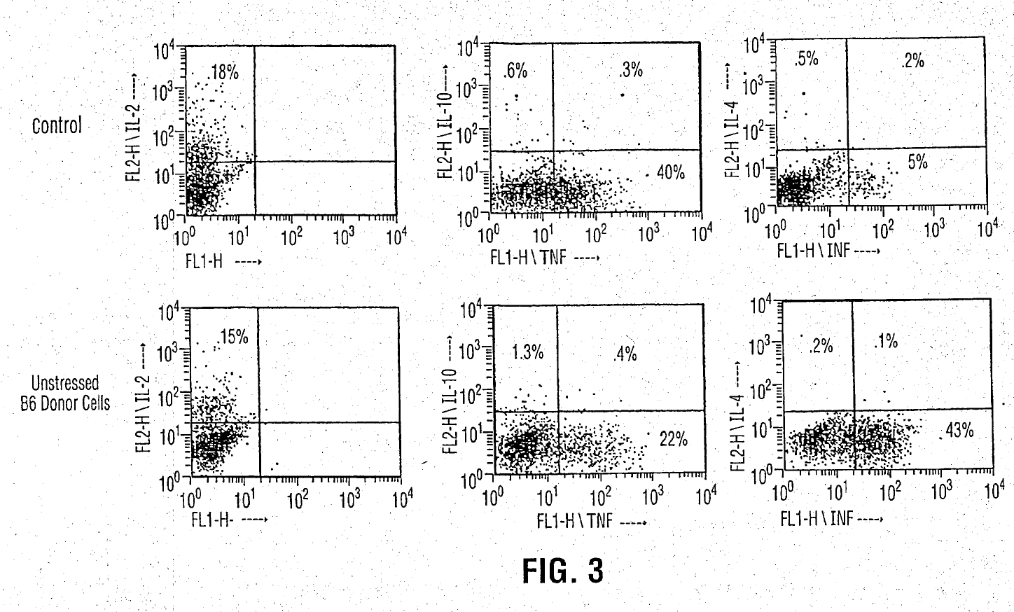

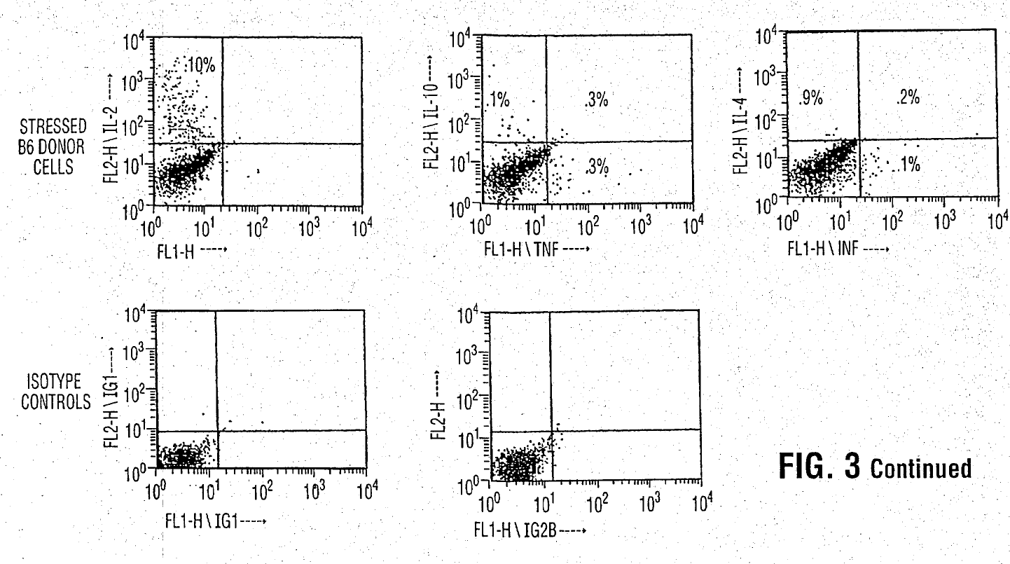

Six days after initiation of GVHD in the mice by injection of the donor cells (treated or untreated), donor T-cells were separated from SCID spleen cells by density gradient centrifugation. Intracellular cytokine staining was performed according to the method of Ferrick, D. A. et. al., NATURE 373 225-257, 1995. The staining was performed on spleen cell suspensions on day 8 after injection of B6 spleen cells. Cytokine production was determined 4 hours after stimulation in vitro with PHA and ionomycin in the presence of brefeldin-A and after gating on CD4⁺ and CD8⁺ cells. The results were assessed by intracellular flow cytometry, and the results thereof are depicted in FIG. 3 of the accompanying drawings. The percentage of each cells in each quadrant is recorded. The drawing shows significantly reduced levels of the inflammatory cytokines interferon-γ (IFN) and tissue necrosis factor-α (TNF), lower right quadrants, from the T-cells which had been stressed as described in Example 1, as compared with untreated cells and controls.

### EXAMPLE 3—COMPARATIVE

Inversion of the normal ratio of CD4+ to CD8+ T-cells (usually approximately 2:1) is known to accompany GVHD. Using anti-CD4 and CD8-tricolor antibodies, CD4/CD8 ratios were determined. In the untreated donor spleen cells after injection into sub-lethally irradiated mice, the inversion of the normal ratio was confirmed. The initial CD4/CD8 ratios of 1.3±0.2 and 2.2±0.3 decreased to 0.33±0.05 and 0.9±0.1 by day 13 for unstressed B6 and C3H donor T cells, respectively, at a time when many animals were succumbing to GVHD. In contrast, the ratios remained greater than 1 at all times and correlated with the lack of clinical evidence of GVHD when donor cells had been pretreated with the stressors as described in Example 1.

### EXAMPLE 4

Human peripheral blood was collected from a consenting donor by venipuncture, and centrifuged to separate the white cell fraction (PBMCs) and the red cell fraction. The PBMCs were washed and re-suspended in autologous plasma, to a concentration of either 3×10⁸ per ml, 1×10⁸ or 5×10⁸ per ml, as shown in the following tables. To some suspensions, there was added 5%, based on the number of PBMCs, of red blood cells (RBCs). The suspensions were then subjected to oxidative stress, heat stress and UV radiation as described in Example 1 above, except for experimental control samples which received no such treatment. Accordingly, there were 4 different groups of samples, as follows:

Group I—3×10⁸ PBMCs+5% RBCs.

Group II—3×10⁸ PBMCs

Group III—1×10⁸ PBMCs+5% RBCs

Group IV—5×10⁸ PBMCs (control).

After subjection to the stressor as described, some of the samples were held in a refrigerator overnight (c.16 hours) at 4° C. The groups were tested for cytokine profile and viability of the T-cell components thereof, by standard staining techniques, immediately after subjection to the stressor, and, in appropriate cases, after the overnight cold storage. In addition, CD4:CD8 ratios were measured for the T-cell components of the various PBMC samples, as described in Example 4.

The results are given in the following Tables 1-4

TABLE 1
The effect of Treatment in accordance with the present invention and
subsequent overnight incubation at 4° C. on PBMC viability

| Before Treatment | 0 hrs after Treatment | % Viable after Treatment |
|---|---|---|
| I - 3 × 10⁸ PBMC + | 1.6 × 10⁷ | 5.3% |
| 5% RBC | | |
| I - 3 × 10⁸ | 1.4 × 10⁷ | 4.3% |
| PBMC | | |
| III - 1 × 10⁸ | 5.0 × 10⁶ | 5.0% |
| PBMC + 5% RBC | | |
| Control - 5 × 10⁸ | N/A | N/A |
| PBMC | | |

TABLE 2
Total Cell Counts in PBMC treated in accordance with
the present invention - Immediate Cultures

| Group No. | After PHA stimulation | After allo-stimulation |
|---|---|---|
| I - 3 × 10⁸ PBMC + 5% | 1.2 × 10⁵ | 8.0 × 10³ |
| RBC | | |
| II - 3 × 10⁸ | 6.0 × 10⁴ | No cells counted |
| PBMC | | |
| Control | 4.7 × 10⁶ | 3.1 × 10⁶ |

TABLE 3
Total Cell Counts in PBMC treated in accordance with the
present invention -Overnight Cultures

| Group No. | After PHA stimulation | After allo-stimulation |
|---|---|---|
| I - 3 × 10⁸ PBMC + 5% | 4.0 × 10⁵ | 5.4 × 10⁴ |
| RBC | | |
| II - 3 × 10⁸ PBMC | 1.4 × 10⁵ | 3.6 × 10⁴ |
| Control | 2.5 × 10⁶ | 3.1 × 10⁶ |

TABLE 4
Intra-Cellular Cytokine Profile in PBMC treated in accordance with the
present invention - Immediate Cultures

| Group No. | IL-2 | TNF-α | CD₄/CD₈ Ratio |
|---|---|---|---|
| I - 3 × 10⁸ PBMC + | 21% | 22% | 76119 |
| 5% RBC | | | |
| Control - 5 × 10⁸ | 52% | 77% | 66/30 |
| PBMC | | | |

The results of these experiments show that human PBMCs have reduced viability immediately following processing in accordance with the present invention to around 4-5% compared to their viability before treatment (Table 1). Cells treated in the presence of 5% RBCs show about a 20% higher viability than cells treated in the absence of RBCs, demonstrating the protective effect of the RBCs.

The results of these experiments also show that human PBMCs have a reduced ability to respond to a number of different stimuli (mitogens, allogeneic cells) immediately following processing in accordance with the present invention (Table 2). However, cells processed in the presence of 5% RBCs show a two-fold greater response to the mitogen PHA and also a greater response to allogeneic stimulation than cells processed in the absence of RBCs. This further confirms the protective effect of RBCs conferred on PBMCs during processing in accordance with the present invention.

Immediately following processing of PBMCs in accordance with the present invention, there is a reduction in the content of the pro-inflammatory cytokines IL2 and TNF of the PBMCs compare to control (untreated) PBMCs. This indicates that human cells react to the stressors in the same way as the murine cells described in the comparative and previously reported examples which also lead to substantial alleviation of GVHD in mice—example 1 above—when injected into experimental animals. Accordingly, human cells so treated can reliably be predicted to be useful in treatment of GVHD in humans, so that they can be administered to patients undergoing BMT, in conjunction with allogeneic stem cells.

### EXAMPLE 5—CLINICAL STUDY

To determine the safety, and to gain indications of the efficacy, of the process of the preferred embodiments of the invention, lymphocytes from 5-6/6 HLA identical donors are treated with oxidative stress and infused into patients diagnosed with acute myelogenous leukemia (AML) with primary non-responsive or relapsed and treatment resistant disease; chronic myelogenous leukemia (CML) in blast crisis refractory to conventional salvage regimens; acute lymphoblastic leukemia (ALL) with primary non-responsive or relapsed and treatment resistant disease; or relapsed, chemo-sensitive non-Hodgkin's lymphoma (NHL) not eligible for auto transplant. Patients have a related donor with a 5-6/6 HLA match, and give informed consent.

Bone marrow is collected from the donor under general anaesthesia; 800-1000 ML of marrow is aspirated from the posterior iliac crests and depleted of T cells by CD34⁺ stem cell selection. CD34 is 110 kD protein expressed on the surface of primitive hematopoietic precursors, endothelial cells and some lymphocytes. The graft is processed in a Miltenyi CLINICMACS separation device according to manufacturer's specifications, a selection system which uses a murine anti-CD34 monoclonal antibody conjugated to paramagnetic beads to bind hematopoietic precursors expressing the antigen. Cells incubated with the antibody are passed over a magnet. The magnet selectively retains hematopoietic precursors while CD34 non-expressing cells, including T cells, are washed free. The magnetic field is then removed and the bound cells which are enriched for hematopoietic precursors with reconstituting activity are washed, eluted, and collected for transplantation. This process uses a closed system to reduce the potential risk of microbial contamination. This procedure results in a 3.5-4 log depletion of lymphocytes. The number of T cells in the positively selected population is determined by flow cytometry. If the number of T cells is greater than 5×10⁴/kg, the separation procedure is repeated until the desired 3.5-4 log depletion is obtained.

Leukopheresis collections from the donor are preformed the day after the bone marrow harvest. Three to four blood volumes are processed on a Cobe SPECTRA® Blood Cell Separator. Venipuncture of peripheral veins is used for venous access, or alternatively, a femoral catheter is placed to allow venous access, if venipuncture is inadequate.

The donor leukapheresis product is collected in a volume of about 300 ml of autologous plasma at a concentration of about 3×10⁷/ml PBMNC's+5% autologous erythrocytes. Ten ml aliquots of the cell suspension are transferred to disposable low-density polyurethane vessels for treatment with a VasoCareÔ medical apparatus (Vasogen Inc., Toronto, Canada), an apparatus as generally described in U.S. Pat. No. 4,968,483 Mueller, et al. During treatment in this apparatus, the sample is heated to and maintained at 42.5° C., while it is oxygenated by exposure to 14.5 μg/ml ozone in oxygen bubbled through the blood cell sample at a flow rate of 240 ml/min, and ultraviolet light at a wavelength of 253.7 nm (0.26 Joules/cm²). The cell suspension is maintained at this temperature during oxygenation for 3 minutes. The entire cycle takes approximately 20 minutes. After removal from the apparatus, the cells are decanted from the polyurethane vessel, immediately washed once in 50 ml of complete medium, and manually counted using a hemocytometer. Cells are suspended in complete medium, adjusted to the desired viable cell concentration in PBS, and then infused into the patient via a central venous catheter.

Acute GVHD is assessed daily while the patient is hospitalized, and then weekly until day 100. It is graded and staged according to the Glucksberg criteria for acute GVHD. Chronic GVHD is assessed and classified according to the Shulman criteria. Detailed immunologic monitoring is performed from day 7 until day 42, and then monthly until the end of the study. The monitoring strategy consists of flow cytometric analysis of T-cell reconstitution; flow cytometric measurement of cytokine production; measurement of proliferative function of reconstituting donor T-cells; measurement of anti-viral and anti-tumor responses by IFNγ-ELISPOT assays; determination of cellular origin by microsatellite analysis, or sex-specific primers in the event of male-female donor-host pairs; and measure of T-cell senescence markers.

What is claimed is:

1. Use in the manufacture of a medicament for administration to a human patient suffering from a bone marrow mediated disease, of an allogeneic population of donor cells containing T-cells cell population which have been subjected, in vitro, to oxidative stress and ultraviolet radiation, in the presence of a biocompatible antioxidant to induce in the allogeneic T-cells an altered cytokine production profile and a reduced proliferative response.

2. Use according to claim 1 wherein the antioxidant is selected from red blood cells, vitamin E, catalase and 2-mercaptoethanol.

3. Use according to claim 2 wherein the biocompatible antioxidant is red blood cells from the donor.

4. Use according to claim 3 wherein the quantity of red blood cells is from about 3-25% by number, based on the number of T-cells which have been subjected to oxidative stress.

5. Use according to any preceding claim wherein the population of donor cells is PBMCs.

6. Use according to any preceding claim wherein the altered cytokine profile of the T-cells is a reduction in production of the anti-inflammatory cytokines INF-γ and TNF-α.

7. Use according to claim 6 wherein the altered cytokine profile of the T-cells additionally comprises an increase in the production of IL-10.

8. Use according to any of claims 1-6 wherein the altered cytokine profile comprises an increase in the production of IL-10.

9. Use according to any of claims 2-9 wherein the allogeneic population of donor cells is enriched in T-cells, and has been subjected to oxidative stress using a gaseous mixture of medical grade ozone and oxygen, bubbled through a suspension of said cells.

10 Use according to claim 9 wherein the T-cell enriched population has been subjected to UV light and oxygen-ozone mixture simultaneously.

11. Use according to claim 10 wherein the T-cell population has been maintained at a temperature of 37-45° C. during subjection to oxidative stress.

12. Use according to any of claims 1-8 wherein the oxidative stress is applied by treating the cells with a chemical oxidizing agent.

13. Use according to claim 12 wherein the chemical oxidizing agent is hydrogen peroxide.

14. A process of treating a mammalian patient for alleviation of a bone marrow mediated disease, with alleviation of consequently developed graft versus host disease (GVHD), which comprises administering to the patient allogeneic hematopoietic stem cells and allogeneic T-cells, at least a portion of said T-cells having been subjected to oxidative stress in vitro, prior to administration to the patient, in the presence of a biocompatible antioxidant so as to induce an altered cytokine production profile and a reduced proliferative response therein.

15. A population of mammalian T-cells, essentially free of stem cells, said T-cells having been subjected in vitro to oxidative stress in the presence of a biocompatible antioxidant, so as to induce in said cells an altered cytokine production profile and a reduced proliferative response.

16. A process of treating allogeneic T-cells, prior to their introduction into a mammalian patient, by extracorporeally stressing the T-cells, which comprises subjecting the T-cells in admixture with red blood cells of the donor, to oxidative stress, to render them suitable for introduction into a mammalian patient undergoing BMT with reduced risk of development of consequent GVHD in said patient.
